(19) United States
(12) Patent Application Publication
(10) Pub. No.: US 2015/0126844 A1
(43) Pub. Date: May 7, 2015

(54) WEARABLE DEVICE WITH LOW ELECTROSTATIC INTERFERENCE

(71) Applicant: Quanta Computer Inc., Kuei Shan Hsiang (TW)

(72) Inventors: Rong-Chin Yang, Kuei Shan Hsiang (TW); Yu-Min Wu, Kuei Shan Hsiang (TW); Yung-Ming Chung, Kuei Shan Hsiang (TW); Chih-Hsiung Yu, Kuei Shan Hsiang (TW)

(73) Assignee: Quanta Computer Inc., Kuei Shan Hsiang (TW)

(21) Appl. No.: 14/170,821

(22) Filed: Feb. 3, 2014

(30) Foreign Application Priority Data
Nov. 6, 2013 (TW) 102140227

(51) Int. Cl. A61B 5/00

(52) U.S. Cl. CPC A61B 5/6823
USPC 600/382

(57) ABSTRACT

A wearable device applied to a human body is provided. The wearable device includes at least one sensing electrode, a conductive wire, a monitor device, and a shield element. The sensing electrode receives a physiological signal from the human body. The sensing electrode is coupled through the conductive wire to the monitor device. The monitor device is configured to process the physiological signal. The shield element includes metal fiber composition. The shield element covers the sensing electrode and the conductive wire so as to avoid electrostatic interference. The shield element is at least partially exposed to the human body.

### CROSS REFERENCE TO RELATED APPLICATIONS

This Application claims priority of Taiwan Patent Application No. 102140227 filed on Nov. 6, 2013, the entirety of which is incorporated by reference herein.

### BACKGROUND OF THE INVENTION

1. Field of the Invention

The disclosure generally relates to a wearable device, and more particularly, relates to a wearable device for detecting human physiological signals and reducing electrostatic interference.

2. Description of the Related Art

With the increase in older people all over the world, there are more and more demands for home care for the elderly. It is an important subject matter for an aging society to provide convenient home-care solutions. Many advanced countries, for example: Germany, Finland, Belgium, Switzerland, and Britain, have invested a large amount of resources to develop a variety of physiological signal monitor devices, which are utilized for monitoring the physiological states of old people and protecting their health and safety at all times. However, when these physiological signal monitor devices are actually applied to human bodies, some problems can occur, such as, electrostatic interference. The aforementioned problem may degrade the quality of physiological signal transmission, and may even lead to some misdiagnoses.

### BRIEF SUMMARY OF THE INVENTION

To overcome the drawback of the prior art, in one exemplary embodiment, the disclosure is directed to a wearable device for use on a human body, including: at least one sensing electrode, receiving a physiological signal from the human body; a conductive wire; a monitor device, wherein the sensing electrode is coupled through the conductive wire to the monitor device, and the monitor device is configured to process the physiological signal; and a shield element, including a fabric with metal fiber composition, and covering the sensing electrode and the conductive wire so as to avoid electrostatic interference, wherein the shield element is at least partially exposed to the human body.

### BRIEF DESCRIPTION OF DRAWINGS

The invention can be more fully understood by reading the subsequent detailed description and examples with references made to the accompanying drawings, wherein:

FIG. 1 is a diagram for illustrating a wearable device according to an embodiment of the invention;

FIG. 2 is a diagram for illustrating a wearable device and a human body according to an embodiment of the invention;

FIG. 3 is a sectional view for illustrating a wearable device and a human body according to an embodiment of the invention;

FIG. 4 is a diagram for illustrating a monitor device according to an embodiment of the invention;

FIG. 5 is a diagram for illustrating a wearable device and a human body according to another embodiment of the invention;

FIG. 6A is a diagram for illustrating a waveform of a measured ECG (Electrocardiography) signal when a shield element is removed from a wearable device; and

FIG. 6B is a diagram for illustrating a waveform of a measured ECG signal when a wearable device includes a shield element, according to an embodiment of the invention.

### DETAILED DESCRIPTION OF THE INVENTION

In order to illustrate the purposes, features and advantages of the invention, the embodiments and figures of the invention are shown in detail as follows.

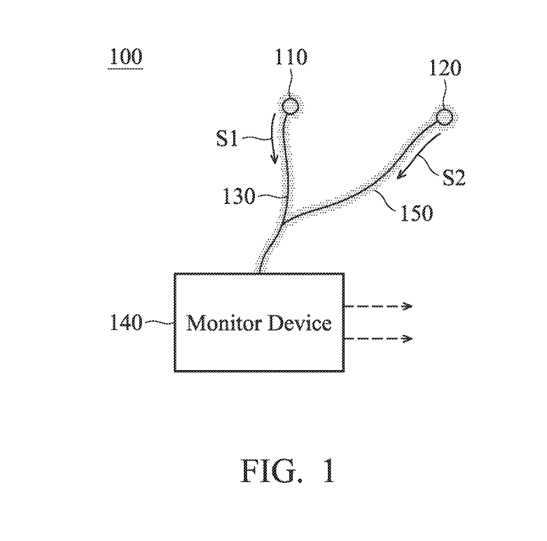

FIG. 1 is a diagram for illustrating a wearable device 100 according to an embodiment of the invention. The wearable device 100 may be worn by a human body, and its type is not limited in the invention. For example, the wearable device 100 may be a cloth, pants, a cap, a bracelet, or other types of accessories. As shown in FIG. 1, the wearable device 100 includes one or more sensing electrodes 110 and 120, a conductive wire 130, a monitor device 140, and a shield element 150. The sensing electrodes 110 and 120 may be directly exposed to the human body, and configured to receive one or more physiological signals S1 and S2 from the human body. For example, the physiological signals S1 and S2 may include heartbeat, blood pressure, breathing rate, body temperature, blood oxygen concentration, muscle contraction, perspiration, and/or EEG (Electroencephalography) waves. The sensing electrodes 110 and 120 are coupled through the conductive wire 130 to the monitor device 140. It is understood that the wearable device 100 may include more sensing electrodes, such as 3, 4, or more than 5 sensing electrodes, in other embodiments although there are merely two sensing electrodes 110 and 120 displayed in FIG. 1.

The monitor device 140 is configured to receive and process the physiological signals S1 and S2, and to wirelessly transmit the processed physiological signals S1 and S2 outwardly. For example, the processed physiological signals S1 and S2 may be wirelessly transmitted by the monitor device 140 to an external device (not shown) for further analysis. The external device may be, for example, a smart phone, a tablet computer, a notebook computer, a desktop computer, or other types of computing devices with wireless communication functions. In other embodiments, adjustments are made such that the monitor device 140 has the functions of storage and analysis, and it can directly store and analyze the physiological signals Si and S2 without transmission to any external device.

The shield element 150 includes metal fiber composition. In some embodiments, the shield element 150 is substantially composed of a soft washable fabric and the metal fiber composition. The shield element 150 may partially or completely cover the sensing electrodes 110 and 120 and the conductive wire 130 so as to avoid electrostatic interference. In a preferred embodiment, the shield element 150 is at least partially exposed to the human body, and it is considered that the shield element 150 is equivalently grounded. The above design can prevent noise from entering the sensing electrodes 110 and 120 and the conductive wire 130, thereby maintaining good quality of physiological signal transmission.

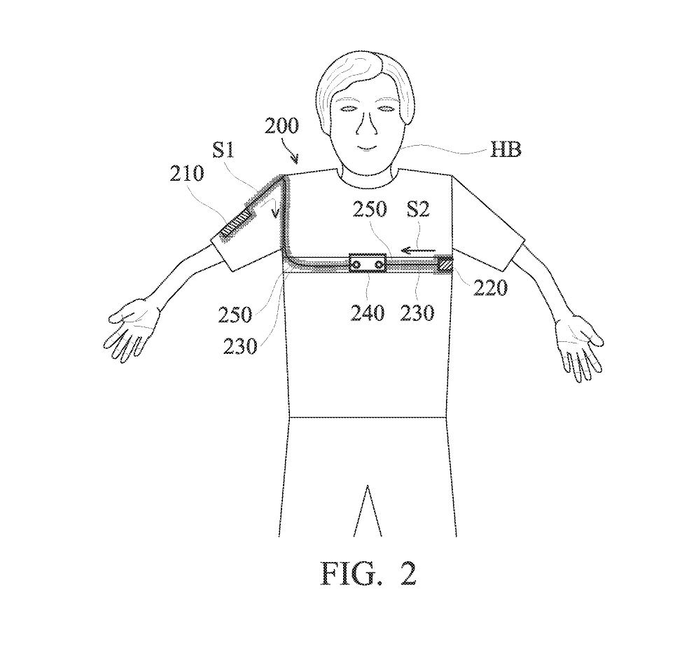

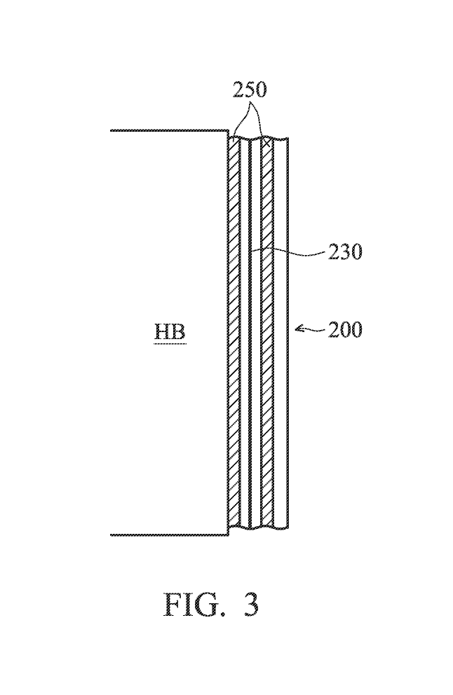

FIG. 2 is a diagram for illustrating a wearable device 200 and a human body HB according to an embodiment of the invention. In the embodiment of FIG. 2, the wearable device 200 is implemented in underwear, which is in close contact with the human body HB. Similarly, the wearable device 200 includes one or more sensing electrodes 210 and 220, a conductive wire 230, a monitor device 240, and a shield element 250. The sensing electrodes 210 and 220 are configured to receive one or more physiological signals S1 and S2 from the human body HB, and are coupled through the conductive wire 230 to the monitor device 240. The monitor device 240 is configured to process the physiological signals S1 and S2, and to wirelessly transmit the processed physiological signals S1 and S2 outwardly. The shield element 250 includes metal fiber composition, and covers the sensing electrodes 210 and 220 and the conductive wire 230. It is understood that the aforementioned components of the wearable device 200 have features similar to those of the wearable device 100 described in the embodiment of FIG. 1. In some embodiments, the sensing electrodes 210 and 220 are conductive textile components, and the conductive wire 230 is a textile cable. The conductive textile components and the textile cable may be appropriately integrated with the underwear so as to form a smart wearable device for detecting the healthy state of the human body HB. The shield element 250 may be a specific fabric. The specific fabric may be made of soft and washable materials, and the metal fiber composition accounts for about 5% to 30% of the special fabric. The ratio of the metal fiber composition keeps good electromagnetic shielding, and it does not result in discomfort of the human body HB when the shield element 250 touches the human body HB. Please further refer to FIG. 3. FIG. 3 is a sectional view for illustrating portions of the wearable device 200 and the human body HB according to an embodiment of the invention. As shown in FIG. 2 and FIG. 3, the shield element 250 covers the sensing electrodes 210 and 220 and the conductive wire 230, and is at least partially exposed to the human body HB. Since the sensing electrodes 210 and 220 and the conductive wire 230 are almost completely separated from other adjacent components (e.g., other outside clothes) by the grounded shield element 250, this design can effectively avoid electrostatic interference and maintain good quality of physiological signal transmission. For example, when the human body HB wears the underwear (i.e., the wearable device 200) and outside perspiration-absorbing clothes to do sport, the shield element 250 can effectively avoid electrostatic interference caused by the friction between the perspiration-absorbing clothes and the underwear. As a result, even if the human body HB takes intense exercise and multiple clothing layers experience friction with each other, the wearable device 200 including the shield element 250 can still correctly receive and process the physiological signals S1 and S2 relative to the human body HB, such that the whole system EMC (Electromagnetic Compatibility) can be improved. It is understood that the statement of “the shield element 250 is at least partially exposed to the human body HB” means that the portion of the shield element 250, covering from the sensing electrode 210 to the monitor device 240 (or from the sensing electrode 220 to the monitor device 240), is partially exposed but partially unexposed to the human body HB.

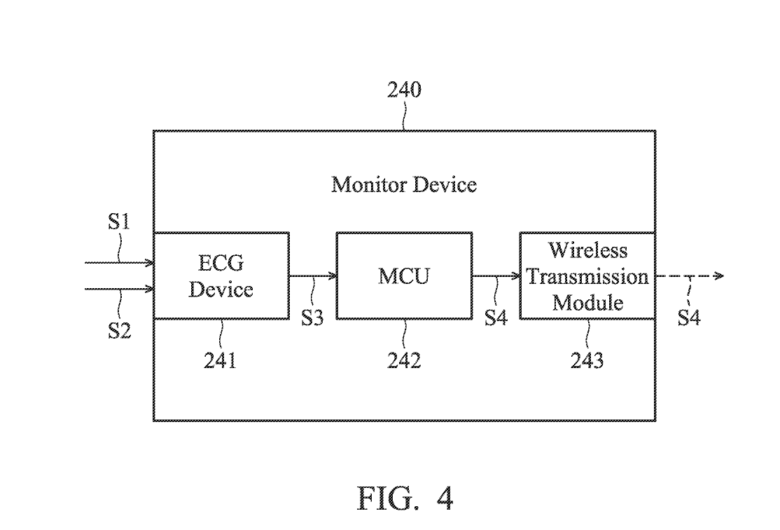

FIG. 4 is a diagram for illustrating the monitor device 240 according to an embodiment of the invention. In the embodiment of FIG. 4, the physiological signals S1 and S2 include heartbeat or pulse, and the sensing electrodes 210 and 220 are respectively adjacent to a right arm and a left armpit of the human body HB. In other embodiments, any one of the sensing electrodes 210 and 220 may be adjacent to other portions of the human body HB, such as a right upper arm, the top of a right armpit, a right armpit, a right chest, or a left chest. As shown in FIG. 4, the monitor device 240 includes an ECG (Electrocardiography) device 241, an MCU (Micro Control Unit) 242, and a wireless transmission module 243. The ECG device 241 is configured to receive the physiological signals S1 and S2, and to convert the physiological signals S1 and S2 into an ECG signal S3. In some embodiments, the ECG device 241 further includes an LNA (Low Noise Amplifier) (not shown) for amplifying the weak physiological signals S1 and S2. The MCU 242 is configured to process and analyze the ECG signal S3, and to generate a digital data signal S4 according to the ECG signal S3. For example, the digital data signal S4 may include a heartbeat frequency and a total number of abnormal events in heartbeat waveforms. The wireless transmission module 243 is further configured to wirelessly transmit the digital data signal S4 to an external device (not shown). For example, the wireless transmission module 243 may be an NFC (Near Field Communication) module, a Bluetooth module, a Wi-Fi module, a 3G module, an LTE (Long Term Evolution) module, or an infrared module. The external device may further analyze the digital data signal S4 so as to obtain the healthy state of the human body HB.

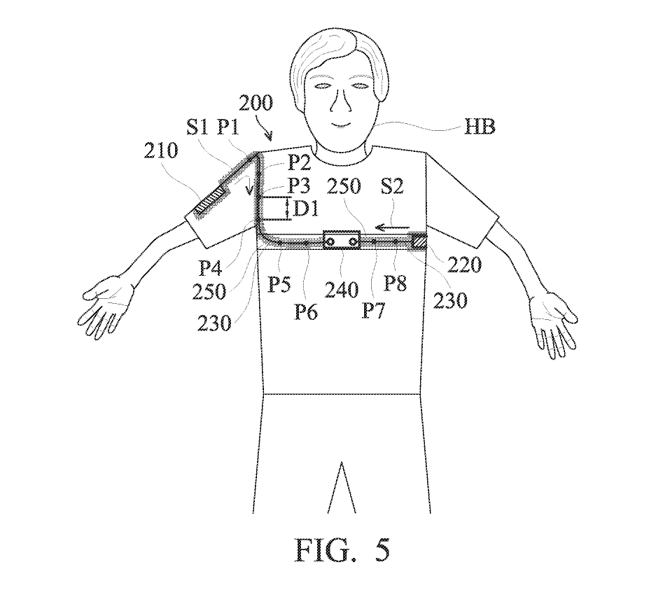

FIG. 5 is a diagram for illustrating the wearable device 200 and the human body HB according to another embodiment of the invention. In the embodiment of FIG. 5, the shield element 250 of the wearable device 200 is discontinuously exposed to the human body HB. More specifically, there are multiple contact points P1 to P8 formed between the shield element 250 and the human body HB, and the shield element 250 does not contact the human body HB except for the aforementioned contact points. To effectively ground the shield element 250 and avoid electrostatic interference, respective spacing D1 between any two adjacent contact points (e.g., P2 and P3, or P3 and P4) selected among the contact points P1 to P8 is designed to be smaller than 10 cm. It is understood that there may be more or fewer contact points formed between the shield element 250 and the human body HB in other embodiments although only eight contact points P1 to P8 are displayed in FIG. 5. In addition, the aforementioned spacing D1 may be further decreased to 5 cm, 3 cm, or 2 cm. In other embodiments, adjustments are made such that the shield element 250 is completely (continuously) exposed to the human body HB so as to achieve the best grounding effect. By arranging the shield element 250 to discontinuously contact the human body HB, the human body HB does not feel so uncomfortable when wearing the wearable device 200.

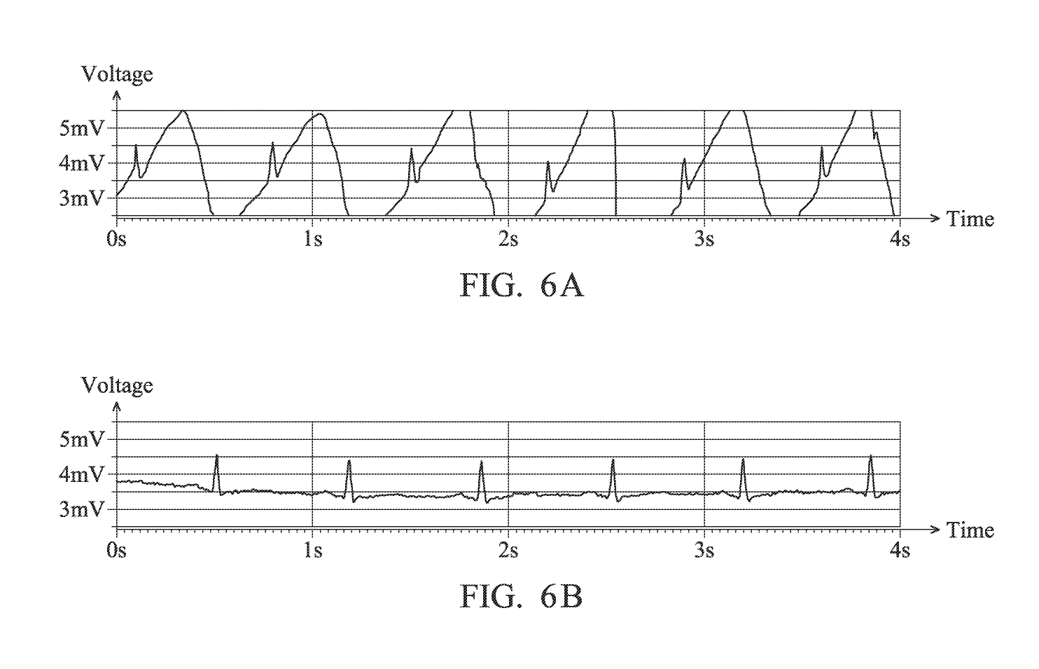

FIG. 6A is a diagram for illustrating a waveform of the measured ECG signal S3 when the shield element 250 is removed from the wearable device 200. In this figure, the horizontal axis represents time (unit: second), and the vertical axis represents voltage (unit: mV). According to the measurement result of FIG. 6A, when the wearable device 200 does not include the shield element 250, the measured ECG signal S3 from the human body HB tends to be affected by electrostatic interference (e.g., the electrostatic interference may be caused by the friction between multiple clothing layers), and it displays an irregular noisy waveform.

FIG. 6B is a diagram for illustrating a waveform of the measured ECG signal S3 when the wearable device 200 includes the shield element 250, according to an embodiment of the invention. In this figure, the horizontal axis represents time (unit: second), and the vertical axis represents voltage (unit: mV). According to the measurement result of FIG. 6B, when the wearable device 200 includes the shield element 250, the electrostatic interference mentioned in FIG. 6A can be effectively eliminated, such that the ECG signal S3 can display a correct and precise heartbeat waveform.

In comparison to the prior art, the invention has at least the following advantages: (1) effectively eliminating electrostatic interference; (2) improving the quality of the physiological signal transmission; (3) correctly displaying the healthy state of the human body; (4) having a simple structure; (5) reducing the discomfort of the user; and (6) decreasing the manufacturing cost. Therefore, the invention may be widely used for a variety of smart wearable devices, and it has commercial production value.

Note that the wearable device is not limited to the configurations shown in FIGS. 1-5. The invention may merely include any one or more features of any one or more embodiments of FIGS. 1-5. In other words, not all of the displayed features in the figures should be implemented in the invention simultaneously.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

It will be apparent to those skilled in the art that various modifications and variations can be made in the invention. It is intended that the standard and examples be considered as exemplary only, with a true scope of the disclosed embodiments being indicated by the following claims and their equivalents.

What is claimed is:

1. A wearable device for use on a human body, comprising:
at least one sensing electrode, receiving a physiological signal from the human body;
a conductive wire;
a monitor device, wherein the at least one sensing electrode is coupled through the conductive wire to the monitor device, and the monitor device is configured to process the physiological signal; and
a shield element, comprising a fabric with metal fiber composition, and covering the at least one sensing electrode and the conductive wire so as to avoid electrostatic interference, wherein the shield element is at least partially exposed to the human body.

2. The wearable device as claimed in claim 1, wherein the at least one sensing electrode comprises two sensing electrodes which are disposed respectively adjacent to a right arm and a left armpit of the human body.

3. The wearable device as claimed in claim 1, wherein the monitor device comprises:
an ECG (Electrocardiography) device, converting the physiological signal into an ECG signal; and
an MCU (Micro Control Unit), generating a digital data signal according to the ECG signal.

4. The wearable device as claimed in claim 3, wherein the monitor device further comprises:
a wireless transmission module, wirelessly transmitting the digital data signal

5. The wearable device as claimed in claim 4, wherein the wireless transmission module is an NFC (Near Field Communication) module, a Bluetooth module, or a Wi-Fi module.

6. The wearable device as claimed in claim 1, wherein the metal fiber composition accounts for about 5% to 30% of the fabric.

7. The wearable device as claimed in claim 1, wherein the shield element is discontinuously exposed to the human body, and a plurality of contact points are formed between the shield element and the human body.

8. The wearable device as claimed in claim 7, wherein spacing between any two adjacent contact points is smaller than 10 cm.

9. The wearable device as claimed in claim 1, wherein the at least one sensing electrode is a conductive textile component, and the conductive wire is a textile cable.

10. The wearable device as claimed in claim 1, wherein the physiological
